(19) United States
(12) Patent Application Publication
(10) Pub. No.: US 2026/0177501 A1
(43) Pub. Date: Jun. 25, 2026

(54) Method for Gating and Clustering and/or Classification of Cell Samples using Multi-Color Assays

(71) Applicant: Sartorius BioAnalytical Instruments, Inc., Bohemia, NY (US)

(72) Inventor: Zhaoping Liu, Rio Rancho, NM (US)

(21) Appl. No.: 18/991,128

(22) Filed: Dec. 20, 2024

(51) Int. Cl. G01N 21/88; G16B 40/20

(52) U.S. Cl. CPC G01N 21/8851; G16B 40/20; G01N 2021/8887

(57) ABSTRACT

The disclosure provides example methods that include obtaining measurements of multiple optical properties (e.g., fluorescent intensities at multiple emission and/or excitation wavelengths) for a plurality of cells from each of a plurality of different cell samples and then gating them into respective sets of two (or more) populations according to each of the optical properties and/or combinations of the optical properties. Virtual cell classes are then determined for each of the cells, with each Boolean combination of populations across all of the optical properties corresponding to a respective different cell class. Counts are determined for each cell class for each sample and normalized within-sample to arrive at a cell class count-based vector characterization of each sample. These vectors can then be displayed to a user (e.g., after clustering along cell classes and/or cell samples) to facilitate the identification of biomarkers or other analysis within the population of samples.

### BACKGROUND

Cell samples can be assessed in a variety of ways, including by individually assessing each cell in the sample (e.g., using a high-throughput flow cytometer). As such sample assessment systems, and the labels used to assess single cells with such systems, become more advanced, the amount of information obtained for each cell can increase. For example, multiple (e.g., more than ten) fluorophores, with respective characteristic emission and/or excitation peaks, can be imaged as independent information channels for each cell, allowing corresponding aspects of the cell (e.g., the presence of an antigen to which a fluorophore is configured to selectively stain, a degree of expression of a gene that codes for or is otherwise associated with a sequence coding for a fluorophore) to be independently measured. This increased information can facilitate additional investigations, e.g., the gating of cells into more cell types more accurately based on the additional channels of information. However, it can be difficult to fully utilize such information, especially when the relationship between the various channels of information and known (or unknown) functional cell types is unknown or unclear.

### SUMMARY

In one aspect, an example method is disclosed. The method includes: (i) obtaining a plurality of cell records for a plurality of cell samples, wherein each cell record of the plurality of cell records represents a set of optical properties detected from a respective cell of one of the cell samples such that each cell sample is associated with a respective non-overlapping subset of the cell records; (ii) for each optical property in the set of optical properties, determining, based on the set of cell records, a respective at least one threshold value that separates the plurality of cell records into at least two populations with respect to the respective optical property; (iii) determining, for each cell sample, a respective set of cell counts, wherein determining a set of cell counts for a particular cell sample comprises determining, for each possible cell class separated by the threshold values, a respective count of cells of the particular cell sample that fall within the respective cell class; and (iv) for each cell sample, normalizing the respective set of cell counts to generate a respective set of normalized cell counts.

In another aspect, an example method is disclosed. The method includes: (i) obtaining a plurality of cell records for a plurality of cell samples, wherein each cell record of the plurality of cell records represents a set of optical properties detected from a respective cell of one of the cell samples such that each cell sample is associated with a respective non-overlapping subset of the cell records; (ii) for each optical property in the set of optical properties, gating, based on the set of cell records, the plurality of cell records into at least two populations with respect to the respective optical property; (iii) determining, for each cell sample, a respective set of cell counts, wherein determining a set of cell counts for a particular cell sample comprises determining, for each possible Boolean logic combination of gated cell records, a respective count of cells of the particular cell sample that fall within the respective Boolean logic combination of gated cell records; (iv) for each cell sample, normalizing the respective set of cell counts to generate a respective set of normalized cell counts; (v) based on the sets of normalized cell counts, clustering at least one of (a) the cell samples according to similarity with respect to their respective sets of normalized cell counts, or (b) the Boolean logic combinations of gated cell records according to similarity with respect to pattern of normalized cell counts across cell samples for each Boolean logic combination of gated cell records; (vi) displaying an indication of the sets of normalized cell counts, ordered according to the clustering; (vii) receiving a user indication of at least one biomarker across the cells samples; and (viii) using the sets of normalized cell counts, training a classifier to predict the at least one biomarker for a cell sample based on an input set of normalized cell counts determined from the cell sample.

In yet another aspect, an example method is provided. The method includes: (i) obtaining a plurality of cell records for a target cell sample, wherein each cell record of the plurality of cell records represents a set of optical properties detected from a respective cell of the target cell sample; (ii) obtaining a plurality of sets of one or more threshold values, wherein a given set of one or more threshold values separates the plurality of cell records into at least two populations with respect to a respective optical property of the set of optical properties; (iii) determining a set of cell counts for the target cell sample by determining, for each possible cell class separated by the threshold values, a respective count of cells of the target cell sample that fall within the respective cell class; (iv) normalizing the set of cell counts to generate a set of normalized cell counts; and (v) applying the normalized cell counts to a trained classifier to predict a biomarker for the target cell sample.

In another aspect, an example non-transitory computer-readable medium is disclosed. The computer readable medium has stored thereon program instructions that upon execution by a processor, cause performance of one or more of the above methods.

In a still further aspect, a system is provided that includes: (i) at least one processor; and (ii) a non-transitory computer-readable medium, having stored therein instructions executable by the at least one processor to cause the system to perform one or more of the above methods. Such a system can include or be in communication with a high-throughput flow cytometer, e.g., to allow the plurality of cell records to be obtained from one or more cell samples by passing some or all of the cell samples through the high-throughput flow cytometer.

The features, functions, and advantages that have been discussed can be achieved independently in various examples or may be combined in yet other examples further details of which can be seen with reference to the following description and drawings.

### BRIEF DESCRIPTION OF THE DRAWINGS

FIG. 1 depicts a distribution of cells from a cell sample with respect to a detected optical property thereof, according to an example implementation;

FIG. 2 depicts the segregation of cells in a cell sample into eight different cell classes based on three detected optical properties thereof, according to an example implementation;

FIG. 3A depicts an example display of normalized cell counts across cell samples and cell classes, according to an example implementation;

FIG. 3B depicts an example display of normalized cell counts across cell samples and cell classes, according to an example implementation;

FIG. 4 depicts an example display of clustered, normalized cell counts across cell samples and cell classes, according to an example implementation;

FIG. 5 is a functional block diagram of a system, according to one example implementation;

FIG. 6 depicts a block diagram of a computing device and a computer network, according to an example implementation;

FIG. 7A shows a flowchart of a method, according to an example implementation;

FIG. 7B shows a flowchart of a method, according to an example implementation;

FIG. 7C shows a flowchart of a method, according to an example implementation.

The drawings are for the purpose of illustrating examples, but it is understood that the inventions are not limited to the arrangements and instrumentalities shown in the drawings.

### DETAILED DESCRIPTION

### I. Overview

Improvements in fluorescent or other biological reporter agents, and the apparatus for detecting them, allow an increasing number of independent channels of optical information to be read out from cell samples (e.g., from individual cells of a sample, using a high-throughput flow cytometer or other laboratory apparatus). These reporter agents can differ with respect to color, excitation and/or emission spectrum, or other optical characteristics to allow them to be detected in a sample in order to measure respective different biological properties of the sample. For example, such a reporter agent could include an antibody or other element that specifically binds to an antigen or other target of interest, allowing the presence, amount or concentration, location, or other information about the target to be detected. In another example, gene(s) coding for such a reporter agent could be introduced into a sample (e.g., into the nuclear DNA of cells of the sample, into some other aspect of the cells of the sample) so that the reporter agent can be detected to allow the amount, spatial distribution, or other information about the expression of the gene(s) (and/or of nearby or otherwise related gene(s) of interest) to be detected.

The ability to separately detect increased numbers of such reporting agents (e.g., fluorophores) or other optical properties of cells means that corresponding increased numbers of cellular substances/processes can be separately detected in a single sample/cell. Such an increase in the amount of optical information detected from a sample is generally beneficial, facilitating additional analyses and investigations and/or reducing the uncertainty with which various cellular processes/contents are detected. For example, increasing the number of detectable cell type-specific antigens and/or ways in which a single such antigen can be detected (e.g., multiple fluorescent reporters configured to selectively bind to respective different epitopes of a single target) can allow the number of cell types and/or sub types that are identifiable within a sample to be increased and/or the certainty with such identifications are made to be increased.

For example, the magnitude of reporter-emitted light or other optical properties detected for a set of antigen-specific reporters or other channels of detected optical information can be used to identify the specific type of an immune cell within a sample. This can include, e.g., applying a branching set of logical gates with respect to the detected reporters, first using two or more reporters (e.g., broadband forward and/or reverse scattering signals, like SSC-H, FSC-H, and/or FSC-A) to gate cells from non-cell particles (e.g., optically active beads) in a sample. The cells identified by the initial gate(s) can then be gated using one or more optical channels to identify live cells (e.g., using a membrane-specific dye, optionally in combination with a forward scatter signal like FSC-A). The identified live cells can then be gated based on returns from an immune-specific reporter (e.g., a fluorophore or other reporter that is detected by the cytometer's fluorescent channel RL2 or is otherwise configured to selectively bind to CD45+ cells) to identify immune cells from the set of live cells. The specific cell types and sub-types of the identified immune cells can then be determined using further optical signals and related gates. For example, B cells could be identified by applying a CD3-specific gate (in the negative direction) followed by a CD19+ gate, while a variety of other sub-types (e.g., CD69+ helper T cells, HLA-DR+ helper T cells, or a composite group of naïve helper T cells and memory cells) could be identified by applying the CD3-specific gate (in the positive direction), followed by a CD4+ gate, and then final subtype-specific gates (e.g., BL6, BL4, and a combination of VL6 and VL5 for CD69+ helper T cells, HLA-DR+ helper T cells, and the composite group of naïve helper T cells and memory cells, respectively).

However, such a specific gating structure, adapted to a specific set of target cell types and sub-types, discards a great deal of the overall optical information that is detected for each detected cell. For example, such a gating structure discards information about the presence of CD4, CD69, HLA-DR, and other antigens in identified B-cells, though that information was generated by the flow cytometer (or other detector apparatus) for those cells (as it was for every detected particle). Further, such gating structures rely upon foreknowledge of the cell types within a cell sample and/or significant manual effort to specify such gating structures for novel or otherwise unknown cell types/populations of cells.

Such shortcomings could be addressed by, e.g., applying machine learning techniques to the full set of optical property data obtained for one or more cell samples. However, the gating functions or other cell-classifying outputs generated by such methods can be difficult to interpret. Additionally, such machine learning techniques are extremely expensive with respect to processor cycles, memory, storage, interconnect bandwidth, power, or other computational costs, and further often require large amounts of training data (e.g., measured optical properties from large numbers of individual cells taken from a large number of cell samples) in order to generate accurate, low-noise, or otherwise useful outputs therefrom. These shortcomings are exacerbated as the number of detected optical properties increases (related, e.g., to the ‘curse of dimensionality’ often observed in the analysis of high-dimensional data).

The methods and other embodiments described herein address these shortcomings, allowing rich data about cell samples, that includes many (e.g., more than a dozen) detected optical properties for each cell thereof, to be used to classify, cluster, or otherwise analyze the cell samples without requiring extensive knowledge about the cell samples. These benefits are obtained without incurring significant computational costs, as would occur when naively applying machine learning techniques (e.g., unsupervised clustering) to such sample data. These benefits are obtained by, for each of the detected optical properties, determining a respective one (or more) thresholds to digitally gate the set of observed cells into a respective set of two (or more) subsets. These automatically (or semi-automatically) generated gates are then used to assign the set of observed cells into every possible cell class separated by the thresholds and/or into every possible Boolean logic combination of subsets with respect to the gates. Once this is done, a respective set of cell counts is determined for each of the cell samples, with the set of cell counts for a particular sample representing the set of counts of each of the cell classes/Boolean logic combinations within the particular sample. These sets of cell counts can then be normalized for each cell sample to generate, for the cell samples, a respective multi-dimensional representation of the cell samples with respect to the distribution of cell classes therein.

Such a method allows a great deal of the complete optical property data measured from a cell sample to be retained and used to characterize the cell sample (as the set of cell counts across cells classes). This benefit is obtained without requiring extensive foreknowledge about the types of cells in the sample and/or extensive manual analysis and annotation to identify such cell types in an unknown or uncharacterized cell sample. Thus benefit is also obtained by the relative computationally inexpensive tasks of separately identifying one (or more) gating thresholds for each of the optical properties and then sorting each of the detected cells into a respective one of the possible cell classes based on separate comparison of each of the cells'optical properties to the corresponding gating threshold(s). This is a significant reduction in computational cost compared to more complex methods of identifying related groups of cells or other structures within the full set of optical property data (e.g., the training of an unsupervised machine learning model, the identification of clusters of cells via k-means or other statistical clustering techniques).

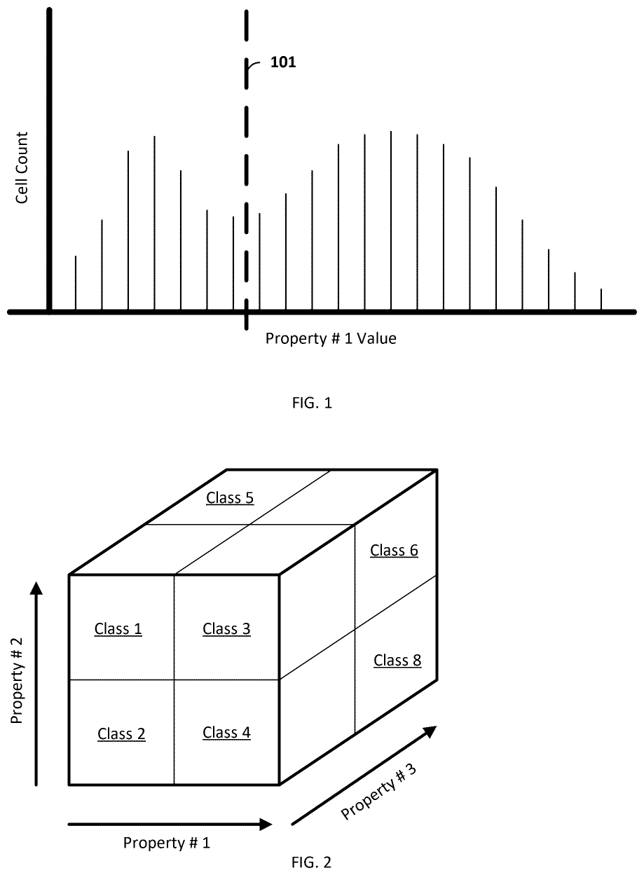

A variety of different methods, or combinations of methods, can be used to determine thresholds to gate populations of cells with respect to a particular measured optical property (e.g., an intensity, duration, or other property of light forward, backward scattered, transmitted, or otherwise interacted-with by a cell, light fluorescently or otherwise emitted by a cell, or light of some other type detected from a cell, e.g., in response to illumination of the cell at the same or a different wavelength than the detected light). By way of an illustrative example, FIG. 1 depicts a histogram of values of a measured optical property across a population of cells (e.g., across cells from a plurality of different cell samples). As shown, the distribution of the optical property across the cells is roughly bimodal, implying that there may be two (or more) sub-populations that exhibit respective different phenotypes that result in respective different exhibited levels of the optical property. Accordingly, it could be beneficial to set a threshold value 101 with respect to the optical property in order to use the measured optical property values to separate the cells into one or the other sub-population.

Note that the illustration of FIG. 1 is intended only as a non-limiting example embodiment; in practice, the distribution of a particular optical property across a population of cells could have more modes (e.g., three modes, four modes), fewer modes (e.g., a single mode), or some other shape (e.g., a relatively uniform distribution in which no single mode is identifiable). More than one threshold could be determined therefrom in order to gate or otherwise partition the set of cells into more than two populations (e.g., two thresholds to gate the cells into three populations corresponding to three modes observed in the distribution). The levels of optical property values depicted in FIG. 1 could represent discrete values of a discrete-valued property (e.g., of a low-resolution detected light intensity) or bins representing ranges of discrete or continuous optical property values.

Determining one (or more) such threshold values (e.g., 101) for a particular optical property can be accomplished in a variety of ways. Methods could include statistical methods that, e.g., fit one, two, or more prototype distributions (e.g., Gaussians) to the observed distribution and then determine the threshold(s) from the parameters of the fitted distributions. For example, a maximum likelihood estimator could be used to determine the threshold value that is most likely to correctly partition samples into the correct population based on a mixture of two fitted distributions that has been estimated from the observed distribution. Additionally or alternatively, pattern analysis methods could be applied to, e.g., identify a local minimum or other feature of a distribution between two maxima (or other estimates of the peaks of the modes of a bimodal distribution) thereof. In some examples, K-means, FlowSom, Phenograph, or some other clustering method can be used to determine the threshold(s) or some other gating structure (e.g., one or more lines, planes, or hyperplanes to gate between multiple populations of cells using two, three, or more optical properties at once) that can then be used to facilitate cell class determination, e.g., via Boolean logical combination. Alternatively, such clustering methods can be used to determine cell class directly. In some examples, the specifics of the threshold(s) or other gating structures could be set or modified manually, using user feedback.

The number of thresholds to determine for a particular optical property could be determined by directly estimating the number of modes or other distinguishable sub-populations within the distribution. Additionally or alternatively, the threshold(s) could be estimated based on several different candidate numbers of sub-populations (e.g., estimating one threshold for two candidate sub-populations, two thresholds for three candidate sub-populations, ...) and some estimate of the goodness of each estimate (e.g., a likelihood of the maximum likelihood estimator used to generate the thresholds) could be used to select one of the candidate numbers of sub-populations and the threshold(s) determined therefor.

Once one or more thresholds have been determined with respect to each of the measured optical properties, the thresholds can be used to gate each observed cell into the appropriate sub-population with respect to each of the measure optical properties. These gatings can then be logically combined to assign each of the observed cells into cell classes such that every possible combination of sub-populations across all of the optical properties is represented by a respective one of the cell classes. This can include determining, for each possible Boolean logical combination of the gates, which of the gated cells fall within the Boolean logical combination of gates.

FIG. 2 depicts, by way of example, the partitioning of a population of cells into a set of such cell classes based on the thresholds determined for each measured optical property. In the example of FIG. 2, three optical properties (“Property #1,” “Property #2,” and “Property #3”) have been measured for a set of cells, and a single threshold determined for each to separate (or gate) the population of cells in to three respective sets of two sub-populations. This partitioning of the population of cells, into two sub-populations with respect to each of three optical properties, results in eight possible cell classes (“Class 1” through “Class 8,” with “Class 7” not shown in FIG. 2). This partitioning can be accomplished by determining, for each cell in the population, which combination of sub-populations across all of the optical properties the cell belongs to. So, for example, a cell whose first optical property measurement is greater than the threshold for the first optical property, whose second optical property measurement is greater than the threshold for the second optical property, whose third optical property measurement is less than the threshold for the third optical property could correspond to “Class 3.” Additionally or alternatively, each such cell class correspond to a respective different Boolean logical combination of cell across all of the gates determined for each of the optical properties.

Note that it is not necessary for a given population to include cell(s) in every such possible cell class. For example, while a population likely includes a non-zero number of cells on either side of the threshold(s) determined for all of the optical properties, it is not necessarily the case that the population include one or more cells in each and every one of the possible Boolean logical combinations of cell gatings across all of the optical properties. E.g., while there is a non-zero number of cells in the set of classes 1, 2, 5 and 7 and in the set of classes 3, 4, 6, and 8 (i.e., the threshold partitioning the population with respect to “Property #1” includes a non-zero number of cells on both sides of the threshold), that does not require there to be a non-zero number of cells in each of the 8 cell classes. Additionally, while FIG. 2 depicts the cell classes by way of geometric volumes of equal size, this is not meant to indicate that the populations of the classes, or on either side of any of the optical property thresholds, are equal, nor that the thresholds are at the “middle” of their respective measured optical property ranges. Yet further, and as noted above, one or more of the optical properties could be partitioned into more than two sub-populations by more than one threshold value. For example, two thresholds could be determined with respect to “Property #1” to partition the population into three sub-populations, resulting in a total twelve cell classes.

As noted above, a cell sample can be characterized by the distribution of cells thereof across such a set of cell classes. Accordingly, a set of cell counts for a cell sample can be determined therefor by counting the number of cells from the cell sample belong to each of the cell classes (i.e., determining a respective cell count, for the cell sample, for each of the cell classes). To account for variation in the total number of cells that may be present in any given cell sample, the cell counts for a sample could then be normalized (e.g., each cell count divided by the total number of cells in the sample). Such a normalization can assist with directly comparing the set of cell counts across difference samples.

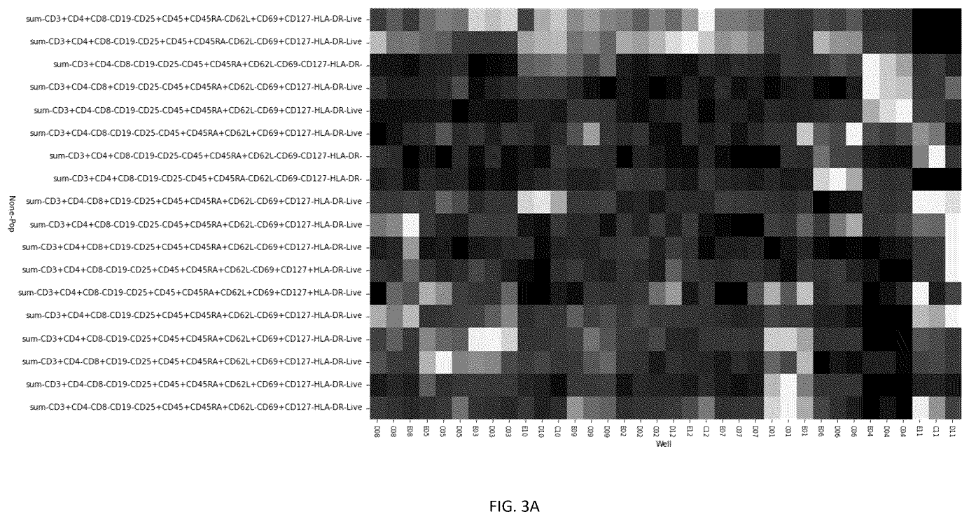

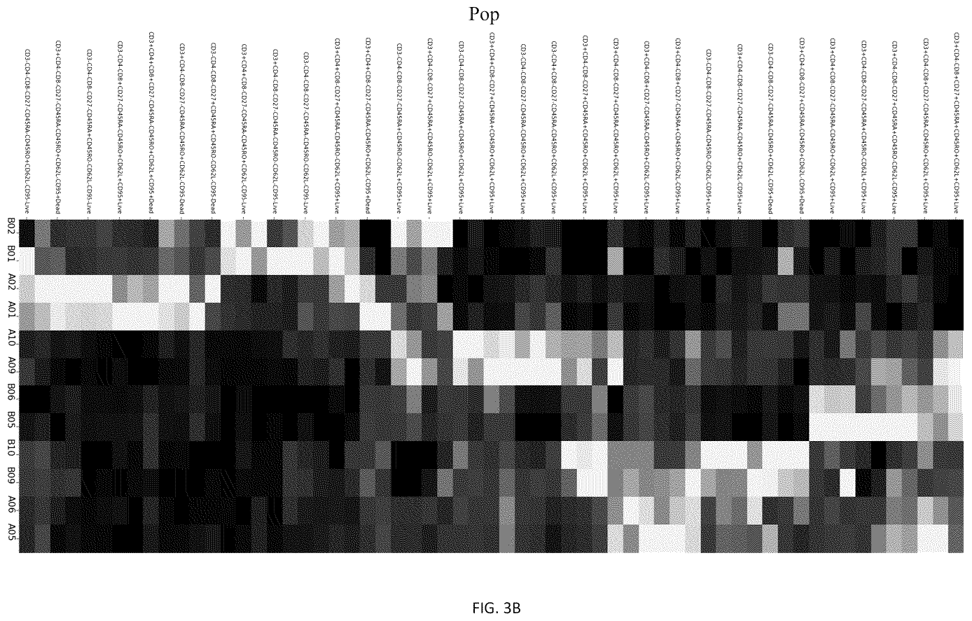

When displaying or otherwise using such sets of cell counts for multiple cell samples (e.g., clustering the cell classes and/or cell samples according to cell count similarity and/or training a classifier to predict a phenotype, biomarker, or other label for a cell sample based on such a set of cell counts), the cell counts make also be normalized across the cell samples, along the cell classes. For example, a maximum count for a given cell class across a set of cell samples could be determined and used to normalize all of the cell counts for the given class across the set of samples (resulting in the maximum count being normalized to a value of ‘1’). FIG. 3A depicts the display of a set of such doubly-normalized cell counts for a set of cell samples (“D11,” “C11,” “E11,” . . . ) and across a number of cell classes (“sum-CD3+ . . . CD62L+ . . . HLA-DR-Live,” “sum-CD3+ . . . CD62L− . . . HLA-DR-Live,” “sum-CD3+CD4− . . . ” . . . ). Such a display can be used to assist a human user in identifying the effects of a treatment or other experimental intervention or difference on the cell samples (e.g., by comparing the pattern of cell counts for a set of samples that received the treatment to the pattern of cell counts for a set of sample that did not), in marking sets of cell samples that appear similar in order to define a potential new biomarker, or to obtain some other benefit.

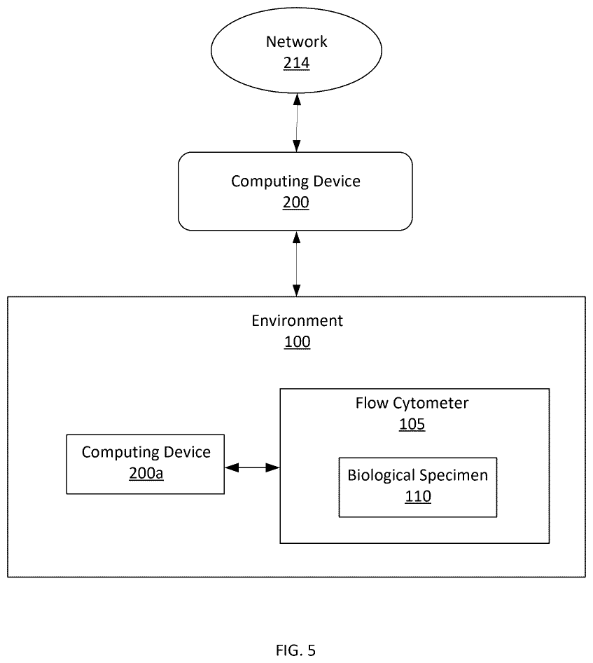

The set of cell counts depicted in FIG. 3A have been generated using the methods described herein, based on ten different measured optical properties (i.e., detected CD3 reporter amplitude, CD4 reporter amplitude, CD8 reporter amplitude, etc.) gated using a single threshold, and thus having two sub-populations for each optical property (indicated in FIG. 3A as ‘+’ or ‘−’). Thus, the methods described herein could be used to determine, for each cell sample, 2\hat{ } or 1024 cell counts for respective cell classes. However, only 18 of the possible 1024 are depicted in FIG. 5A. This is due to many of the non-displayed cell classes having been removed due to some disqualifying factor, e.g., come of the classes having zero total cell counts and/or zero or otherwise subthreshold (or super-threshold) cell counts with respect to one or more cell samples.

Such disqualifying factors could include one or more of the cell counts for a particular cell class in a single cell sample, or across all cell samples combined, being less than, greater than, or otherwise exceeding some absolute or relative cell count level. For example, if the cell count for a particular cell class is equal to zero for any of the cell samples and/or if the combined cell count for a particular cell class across all of the cell samples is equal to zero (or beyond some other user-specified threshold), that particular cell class could be disqualified from display and/or from some other analysis (e.g., clustering, training a classifier or other machine learning model). This could be done to avoid including classes that are noisy, that are not well-represented in all of the cell samples, or that might otherwise deleteriously affect some downstream analysis or display. Additionally or alternatively, cell classes that exhibit too-high cell counts with respect to one or more cell samples could be disqualified, e.g., due to such over-representation diminishing the utility of such a cell class in discriminating the differences between cell samples. The disqualification of cell classes could also be motivated by a desire to emphasize the ‘most valuable’ cell classes for display in a limited display area (e.g., where human perception or preferences specify a minimum area to indicate a single cell count, so a finite display can only display a correspondingly finite number of cell classes, motivating a winnowing of the cell classes to the ‘most valuable’).

Once the determination of normalized cell counts (e.g., doubly normalized, along cell samples and along optical properties) for one or more cell samples has been accomplished according to the methods described herein, such normalized cell counts can be used to accomplish various downstream displays, analyses, or other ends. For example, the normalized cell counts for a number of cell samples could be provided for display to a human user (e.g., as depicted in FIG. 3A) to allow the human user to more readily apprehend the large amounts of optical property data determined for the cell samples. This can enable the user to, e.g., identify or investigate the presence of biomarkers within the data that may be relevant to the presence, prognosis, treatment, or other properties of a disease, syndrome, phenotype, or other biological process or factor.

Identification of clinically or scientifically relevant biomarkers (or other patterns or contents of the displayed cell count data) can be facilitated by co-display of relevant information (e.g., indications of whether a cell sample represents a treatment or control group) and/or by performance of some level of analysis on the cell count data prior to display. For example, the data could be clustered with respect to the cell samples (i.e., such that cell samples with similar sets of cell counts are displayed nearer to each other than to samples with dissimilar sets of cell counts) and/or with respect to the optical properties (i.e., such that optical properties corresponding to similar sets of cell counts across the cell samples are displayed nearer to each other than to optical properties that correspond to dissimilar sets of cell counts across the cell samples). By performing such a clustering, and then displaying the data based on the clustering (e.g., such that cell samples that have been clustered together are adjacent in the display or otherwise nearby each other), patterns of similarity within the data (e.g., biomarkers or other common patterns in the cell count data across cell samples and/or optical properties) could be more readily apprehended by a user. This could aid the user in, e.g., identifying one or more biomarkers within the data that are represented by common cell count patterns across multiple cell samples.

FIG. 3B depicts the display of a set of doubly-normalized cell counts for a set of cell samples (“B02,” “B01,” “A02,” . . . ) and across a number of cell classes (“CD3+ . . . CD27+ . . . Live,” “CD3+ . . . CD27− . . . Live,” . . . ) wherein the data has been clustered with respect to both cell sample and optical property prior to display. Such a display can be used to assist a human user in identifying one or more potential biomarkers within the data or to obtain some other benefit. For example, a user could identify four (or more) potential biomarkers within the data based on similarities in the patterns of cell count data along the optical properties for respective four subsets of the cell samples (e.g., a first subset including the first four samples “B01” to “A01,” a second subset including “A10” and “A09,” a third subset including “B06” and “B06,” and a fourth subset including “B10” through “A05”). The identification of such biomarkers could facilitate the identification or development of corresponding distinct phenotypes (e.g., of a person, animal, or disease), disease mechanisms, patterns of response to a treatment or other intervention, types or sub-types of a disease (e.g., of a cancer), diagnostics to detect a disease or other condition by detecting the biomarkers, patient stratification for treatment, application of precision medicine, or of some other treatments, diagnostics, interventions, or other applications.

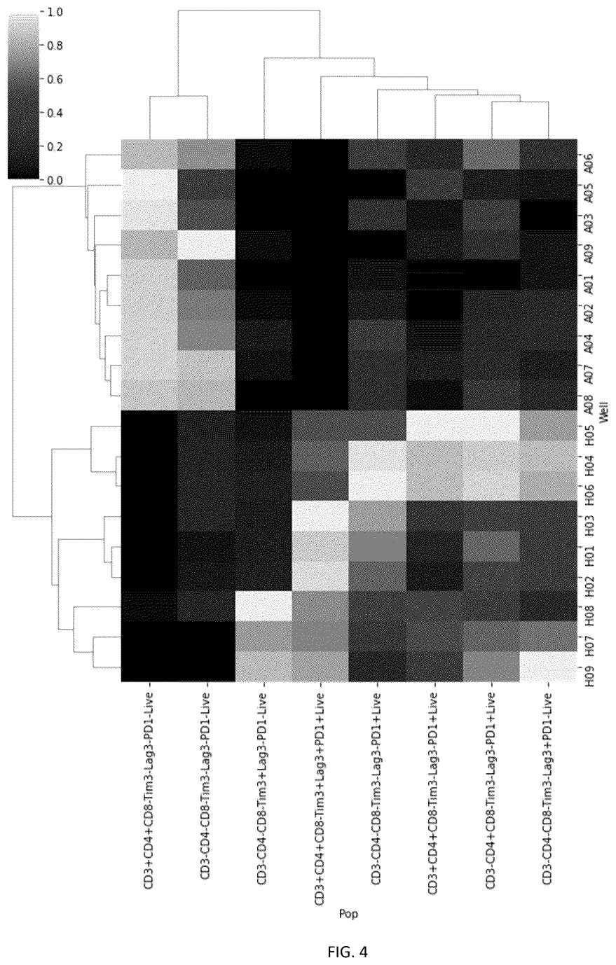

Such clustering may be performed via a variety of methods, and may be performed with or without the various pre-processing steps described herein (e.g., without normalizing the cell counts along the optional properties, without removing optical properties that are under-/over-represented in one or more of the cell samples). The clustering could be a hierarchical clustering, e.g., using single-linkage, ward-linkage, centroid-linkage, median-linkage, or some other linkage method, using Euclidean, maximum, Manhattan, Canberra, binary, Minkowsky, or some other metric method, or some other hierarchical clustering method. The clusters or other structure determined by the clustering (e.g., the pattern of a hierarchical clustering) could be displayed with the cell counts, in order to further inform a user. For example, a dendrogram could be displayed with the cell count data in order to indicate the pattern of hierarchical clusters within a hierarchical clustering. FIG. 4 depicts an example of such a display, wherein dendrograms are indicated to the side of a display of cell count data in order to indicate the hierarchical pattern of clusters determined for the cell count data with respect to both similarities with respect to cell count patterns between cell samples (the dendrogram to the side) and with respect to similarities between patterns of cell counts across optical properties (the top dendrogram).

Sets of cell counts as determined herein (e.g., with or without normalizing the cell counts along the optional properties, with or without removing optical properties that are under-/over-represented in one or more of the cell samples) could additionally or alternatively be used to train a classifier or other machine learning model to make a prediction about a cell sample based on the set of cell counts therefor. This could include predicting a disease state or performing some other diagnosis for the cell sample, determining a phenotype for the cell sample, predicting a biomarker for the cell sample, or performing some other analysis of the cell sample based on the set of cell counts.

Training a classifier or other machine learning model to output such predictions can include obtaining a training dataset that includes: i) sets of cell counts as described herein for respective cell samples, and ii) labels for the cell samples indicating the aspect of the samples that is to be predicted by the model. For example, if the predicted output is the presence of a disease or other condition, the labels could indicate whether each cell sample was obtained from a person who had the disease or condition, or not. The labels could be discrete-valued (e.g., yes/no for the presence or absence of a condition or other binary state, one of an enumerated set of possible values for a condition having a limited number of possible states) or continuous-valued (e.g., to predict the degree of presence of a continuously-varying process or factor, to predict a likelihood that a binary state is or is not true). For example, the predicted output could be the presence of a biomarker, where the biomarker is a pre-specified pattern within the cell counts across the optical properties.

The training data could be obtained from user inputs. For example, a user could indicate a subset of cell samples as corresponding to a particular biomarker of interest (and, optionally, another subset of the cell samples that does not correspond to the biomarker of interest). This indication can be converted into labels for all of the cell samples (with the labels for the indicated subset representing the presence of the biomarker, and absence in the other cell samples) and the labels, in combination with the cell count data, used to train the classifier or other machine learning model. In some examples, the results of a clustering operation (e.g., that has been performed to facilitate manual identification of biomarkers within the data) could be provided as an auxiliary training input to the model. For example, an indication of the pattern of a hierarchical clustering could be provided in order to assist the model in identifying potentially relevant structure within the data, thereby reducing the computational cost to train the model by, e.g., allowing the model to reach a desired level of accuracy in fewer training iterations or steps.

A classifier or other machine learning model trained in this manner can then be operated to generate predictive outputs for novel cell samples based on sets of cell counts detected therefor. For example, to detect the presence of a biomarker pattern in the cell counts for a cell sample, to diagnose a disease state from the cell counts for a cell sample, to determine a phenotype of a cell samples, or to determine some other output for a cell sample. Such outputs can then be used to achieve some benefit, e.g., by providing a treatment if a disease state is detected, by selecting a specific type of drug or otherwise tailor a course of treatment to a detected biomarker or phenotype of a cell sample, or by performing some other act.

In order to use a trained machine learning model in this manner, the set of cell counts for a target sample can be determined in the same manner as was used to generate the cell count data used to train the model. So, the sets of optical properties for each cell in the target sample can be obtained (e.g., by operating a high-throughput flow cytometer) and then used to determine cell class-specific cell counts by applying the determined threshold(s) for each optical property to gate the cells and then assigning the cells to the appropriate cell class according to the gating. The cell counts for the target cell sample can then be normalized. If the cell count data used to train the model was doubly normalized, a set of further scaling factors could then be applied to the target sample cell counts, in order to scale them in the same manner that the cell counts in the training data were scaled in order to normalize the cell counts along each of the optical properties. If a subset of the cell classes were discarded (e.g., due to one or more of the training cell samples not including any instances of one or more of the cell classes), the corresponding cell counts for the target sample could also be discarded prior to applying the target sample cell counts to the training model, in order to correspond the number and identity of the input cell counts to that of the training data.

Optical property data obtained for the cell of a cell sample as described herein could include a variety of different optical properties obtained in a variety of different ways, e.g., using a high-throughput flow cytometer. Such optical properties could include the magnitude, intensity, amplitude, width, shape, or other properties of a time-varying pulse of light at one or more wavelengths that is received from (e.g., emitted or scattered by) the cell in response to illumination at one or more wavelengths of light. For example, one or more of the detected optical properties could be a height and/or width of a pulse of light forward, side, and/or reverse scattered by a cell in one or more specific direction or ranges of directions relative to a direction from which illumination (e.g., white light illumination, illumination at one or more specified wavelengths) is projected toward the cell. In some examples, one or more of the optical properties could be an intensity (or other measured property) of light emitted from the cell at a first specified excitation wavelength in response to illumination at a second specified emission wavelength. The cell can be dyed, genetically modified, or otherwise prepared such that it includes a fluorophore that, in response to excitation at the first wavelength, fluorescently emits light at the second wavelength. The cell could include a plurality of dye fluorophores or other dyes, which could differ with respect to the excitation and/or emission wavelengths in order to facilitate multiplexed detection of many such reporter compounds in a single cell. In this manner, the number of optical properties that can be detected from a single cell (e.g., during the limited period of time that the single cell is able to be measured by a flow cytometer) can be increased, e.g., to dozens of optical properties. As noted above, where such large numbers of detected optical properties are available for a sample of interest, the methods described herein can provide significant benefits with respect to the computational analysis thereof.

### II. Example Architecture

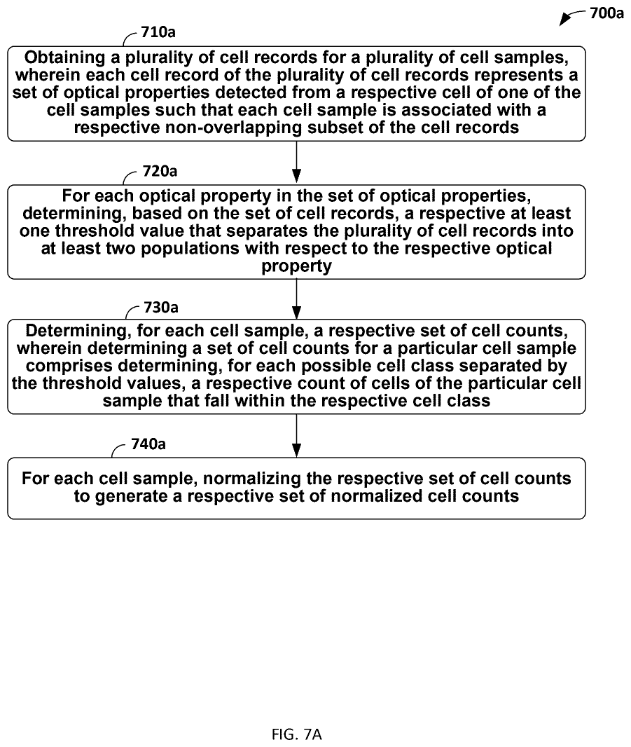

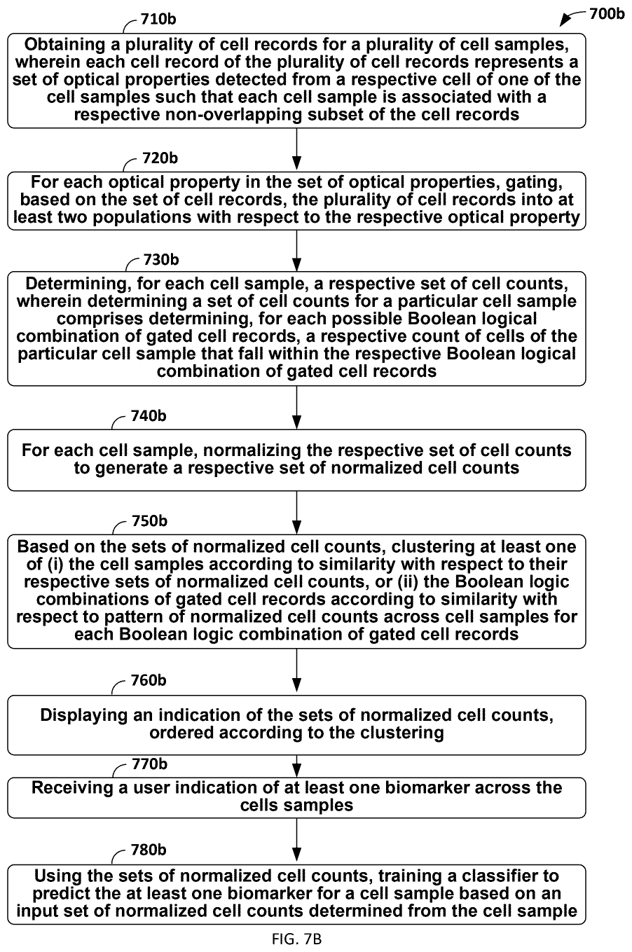

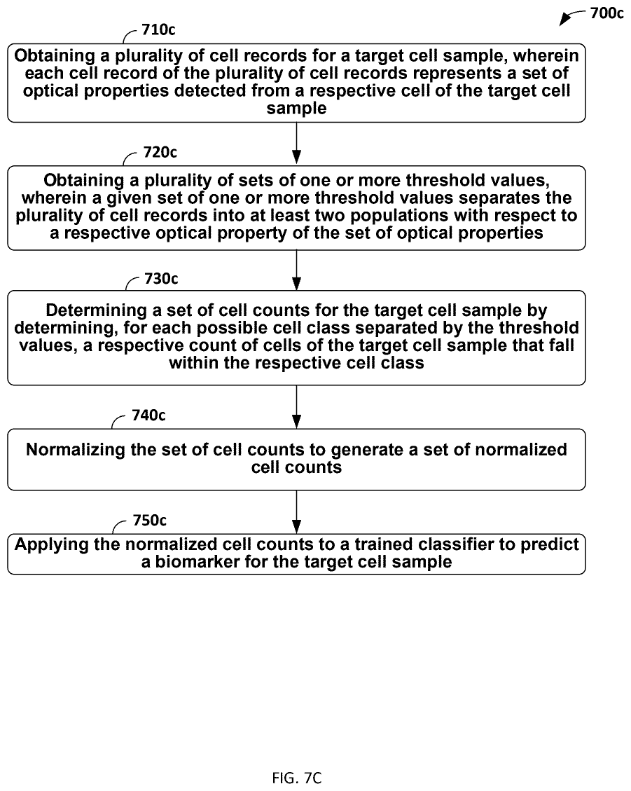

FIG. 5 is a block diagram showing an operating environment 100 that includes or involves, for example, a high-throughput flow cytometer 105 and a biological specimen 110 having a plurality of more cells. Methods 700a-c in FIGS. 7A-C described below show embodiments of method that can be implemented within this operating environment 100.

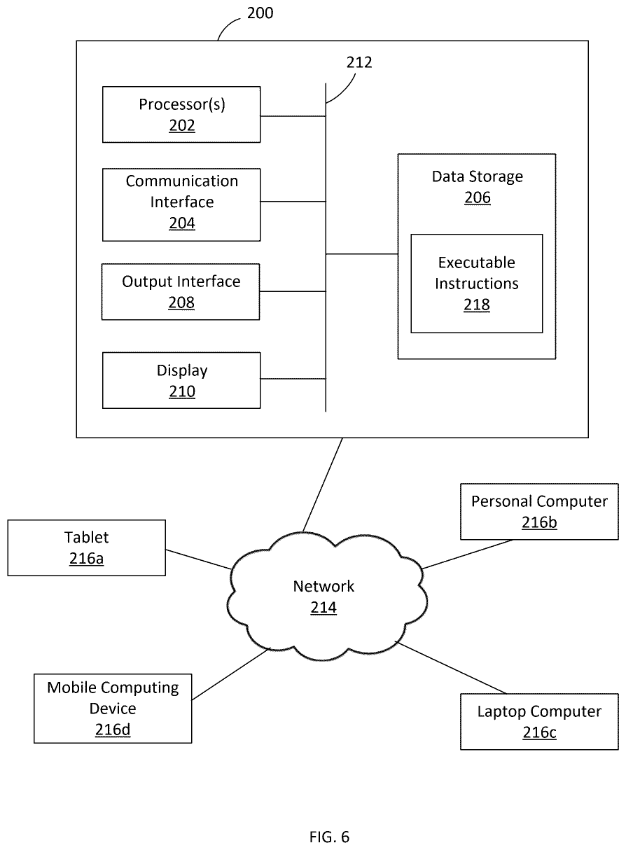

FIG. 6 is a block diagram illustrating an example of a computing device 200, according to an example implementation, that is configured to interface with operating environment 100, either directly or indirectly. The computing device 200 may be used to perform functions of methods described herein, e.g., those shown in FIGS. 7A-C and described below. Computing device 200 can be configured to perform one or more functions, including cell gating, counting, clustering, normalization, classification, model training, display, or other functions that are based, in part, on sets of optical properties of cells obtained by the high-throughput flow cytometer 105, for example. The computing device 200 has a processor(s) 202, and also a communication interface 204, data storage 206, an output interface 208, and a display 210 each connected to a communication bus 212. The computing device 200 may also include hardware to enable communication within the computing device 200 and between the computing device 200 and other devices (e.g. not shown). The hardware may include transmitters, receivers, and antennas, for example.

The communication interface 204 may be a wireless interface and/or one or more wired interfaces that allow for both short-range communication and long-range communication to one or more networks 214 or to one or more remote computing devices 216 (e.g., a tablet 216a, a personal computer 216b, a laptop computer 216c and a mobile computing device 216d, for example). Such wireless interfaces may provide for communication under one or more wireless communication protocols, such as Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wired interfaces may include Ethernet interface, a Universal Serial Bus (USB) interface, or similar interface to communicate via a wire, a twisted pair of wires, a coaxial cable, an optical link, a fiber-optic link, or other physical connection to a wired network. Thus, the communication interface 204 may be configured to receive input data from one or more devices, and may also be configured to send output data to other devices.

The communication interface 204 may also include a user-input device, such as a keyboard, a keypad, a touch screen, a touch pad, a computer mouse, a track ball and/or other similar devices, for example.

The data storage 206 may include or take the form of one or more computer-readable storage media that can be read or accessed by the processor(s) 202. The computer-readable storage media can include volatile and/or non-volatile storage components, such as optical, magnetic, organic or other memory or disc storage, which can be integrated in whole or in part with the processor(s) 202. The data storage 206 is considered non-transitory computer readable media. In some examples, the data storage 206 can be implemented using a single physical device (e.g., one optical, magnetic, organic or other memory or disc storage unit), while in other examples, the data storage 206 can be implemented using two or more physical devices.

The data storage 206 thus is a non-transitory computer readable storage medium, and executable instructions 218 are stored thereon. The instructions 218 include computer executable code. When the instructions 218 are executed by the processor(s) 202, the processor(s) 202 are caused to perform functions. Such functions include, but are not limited to, operating the flow cytometer 105 to obtain respective sets of optical properties (e.g., magnitudes, width, or other properties of forward, reverse, and/or side-scattered light at one or more wavelengths, fluorescently or otherwise emitted light at one or more wavelength when illuminated by light at one or more different wavelengths, or other light detected from cells) of cells from the biological specimen 110. Such functions could additionally or alternatively including functions to determine one or more threshold values to gate cells into one or more sub-populations with respect to each such measured optical property, to count the number of cells that are present in a cell sample by cell class, to cluster, classify, display, or otherwise analyze one or more cell samples according to the set of cell class cell counts determined therefor, to train a classifier or other machine learning model to predict a phenotype, biomarker, or other label for a cell sample based on a set of cell class cell counts, and/or to perform some other computation as described herein.

The processor(s) 202 may be a general-purpose processor or a special purpose processor (e.g., digital signal processors, application specific integrated circuits, etc.). The processor(s) 202 may receive inputs from the communication interface 204, and process the inputs to generate outputs that are stored in the data storage 206 and output to the display 210. The processor(s) 202 can be configured to execute the executable instructions 218 (e.g., computer-readable program instructions) that are stored in the data storage 206 and are executable to provide the functionality of the computing device 200 described herein.

The output interface 208 outputs information to the display 210 or to other components as well. Thus, the output interface 208 may be similar to the communication interface 204 and can be a wireless interface (e.g., transmitter) or a wired interface as well. The output interface 208 may send commands to one or more controllable devices, for example

The computing device 200 shown in FIGS. 5 and 6 may also be representative of a local computing device 200a in operating environment 100, for example, in communication with high-throughput flow cytometer 105. This local computing device 200a may perform one or more of the steps of the method(s) 700a-c described below, may receive input from a user and/or may send cell count and/or optical property data and user input to computing device 200 to perform all or some of the steps of method(s) 700a-c.

FIGS. 7A-C show flowcharts of example methods 700a-c to determine, for one or more cell samples, respective sets of cell counts across a set of cell classes, e.g., to generate such a set of cell counts for the biological specimen 110, according to an example implementation. Methods 700a-c shown in FIGS. A-C present exemplary methods that can be used with the computing device 200 of FIG. 6, for example. Additionally or alternatively, some or all of the functionality of the methods described herein (e.g., methods 700a-c) could be performed by the servers, processors, or other elements of a cloud computing service, remote server, or other computing system that is remote from but in communication with the computing device 200 and/or flow cytometer 105 (and optionally additional such computing devices and/or flow cytometers) via the network 214 (e.g., via the Internet). This could be beneficial in that such a remote computing system could have more extensive storage, database systems, memory, processing resources, or other computing resources to facilitate the determination of optical property-specific thresholds, determining (and optically normalizing) cell counts across large numbers of cell classes for large numbers of cell samples, clustering such data by cell class and/or cell sample, training classifiers or other machine learning systems to operate on such cell count data (e.g., to predict one or more biomarkers from a set of cell samples, to predict whether a target cell sample has a particular phenotype, exhibits a biomarker, or corresponds to some other label or property), or performing some other computation or process as described herein. Such a remote (e.g., cloud-based) computing system could also have the benefit of access to many flow cytometers or other sources of flow cytometry data, allowing the remote system to train improved classifiers or other machine learning models or to otherwise improve the performance of the methods described herein.

Further, devices or systems may be used or configured to perform logical functions presented in FIG.(s) 7A-C. In some instances, components of the devices and/or systems may be configured to perform the functions such that the components are configured and structured with hardware and/or software to enable such performance. Components of the devices and/or systems may be arranged to be adapted to, capable of, or suited for performing the functions, such as when operated in a specific manner. Methods 700a-c may include one or more operations, functions, or actions as illustrated by one or more of the blocks thereof. Although the blocks are illustrated in a sequential order, some of these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of the present examples. In this regard, each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time such as register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block in each of FIGS. 7A-c, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

### III. Example Methods

Referring now to FIG. 7A, a method 700a is illustrated, optionally using the computing device of FIGS. 5-6. Method 700a includes, at block 710a, obtaining a plurality of cell records for a plurality of cell samples, wherein each cell record of the plurality of cell records represents a set of optical properties detected from a respective cell of one of the cell samples such that each cell sample is associated with a respective non-overlapping subset of the cell records. Then, at block 720a, the method 700a includes, for each optical property in the set of optical properties, determining, based on the set of cell records, a respective at least one threshold value that separates the plurality of cell records into at least two populations with respect to the respective optical property. Next, at block 730a, the method additionally includes determining, for each cell sample, a respective set of cell counts, wherein determining a set of cell counts for a particular cell sample comprises determining, for each possible cell class separated by the threshold values, a respective count of cells of the particular cell sample that fall within the respective cell class. The method 700a then includes, for each cell sample, normalizing the respective set of cell counts to generate a respective set of normalized cell counts, at block 740a.

The method 700a can additionally include: (i) based on the sets of normalized cell counts, clustering at least one of (a) the cell samples according to similarity with respect to their respective sets of normalized cell counts, or (b) the cell classes according to similarity with respect to pattern of normalized cell counts across cell samples for each cell class; and (ii) displaying an indication of the sets of normalized cell counts, ordered according to the clustering. Such a method could additionally include, prior to displaying the indication, further normalizing the sets of normalized cell counts within each of the cell classes. Additionally or alternatively, such a method could additionally include: (i) receiving a user indication of one or more subsets of samples within the plurality of cell samples; and (ii) using the sets of normalized cell counts, training a classifier to predict membership of a cell sample within the one or more subsets of samples based on an input set of normalized cell counts determined from the cell sample. Additionally or alternatively, such a method could additionally include, prior to displaying the indication, removing, from the cell classes, any of the cell classes that are represented in any of the cell samples by a cell count that exceed a threshold value. In such a method, clustering at least one of (i) the cell samples according to similarity with respect to their respective sets of normalized cell counts, or (ii) the cell classes according to similarity with respect to pattern of normalized cell counts across cell samples for each cell class could include performing a hierarchical clustering.

The method 700a can additionally include: (i) obtaining a set of labels that identify one or more subsets of samples within the plurality of cell samples; and (ii) using the sets of normalized cell counts and sets of labels, training a classifier to predict membership of a target cell sample within the one or more subsets of samples based on an input set of normalized cell counts determined from the target cell sample. Such a method could further include, prior to training the classifier, further normalizing the sets of normalized cell counts within each of the cell classes. Additionally or alternatively, such a method could further include, prior to training the classifier, removing, from the cell classes, any of the cell classes that are represented in any of the cell samples by a cell count that is less than a threshold value. Additionally or alternatively, such a method could further include, prior to training the classifier and based on the sets of normalized cell counts, clustering at least one of (i) the cell samples according to similarity with respect to their respective sets of normalized cell counts, or (ii) the cell classes according to similarity with respect to pattern of normalized cell counts across cell samples for each cell class.

In the method 700a, determining, based on the set of cell records, a respective at least one threshold value that separates the plurality of cell records into at least two populations with respect to the respective optical property could include, for a particular optical property of the set of optical properties, determining at least two threshold values that separates the plurality of cell records into at least three populations with respect to the particular optical property.

In the method 700a, a first optical property of the set of optical properties could represent an intensity of light at a first wavelength that is received from a cell in response to the cell being illuminated by light at a second wavelength, and a second optical property of the set of optical properties could represent an intensity of light at the first wavelength that is received from a cell in response to the cell being illuminated by light at a third wavelength that differs from the second wavelength.

The method 700a could additionally include operating a high-throughput flow cytometer to obtain the plurality of cell records for the plurality of cell samples.

The method 700a could include additional or alternative steps or features.

Referring now to FIG. 7B, a method 700b is illustrated, optionally using the computing device of FIGS. 5-6. Method 700b includes, at block 710b, obtaining a plurality of cell records for a plurality of cell samples, wherein each cell record of the plurality of cell records represents a set of optical properties detected from a respective cell of one of the cell samples such that each cell sample is associated with a respective non-overlapping subset of the cell records. Then, at block 720b, the method 700b includes, for each optical property in the set of optical properties, determining, based on the set of cell records, a respective at least one threshold value that separates the plurality of cell records into at least two populations with respect to the respective optical property. Next, at block 730b, the method 700b additionally includes determining, for each cell sample, a respective set of cell counts, wherein determining a set of cell counts for a particular cell sample comprises determining, for each possible Boolean logical combination of gated cell records, a respective count of cells of the particular cell sample that fall within the respective Boolean logical combination of gated cell records. The method 700b then includes, for each cell sample, normalizing the respective set of cell counts to generate a respective set of normalized cell counts, at block 740b. At block 750b, the method 700b includes, based on the sets of normalized cell counts, clustering at least one of (i) the cell samples according to similarity with respect to their respective sets of normalized cell counts, or (ii) the Boolean logic combinations of gated cell records according to similarity with respect to pattern of normalized cell counts across cell samples for each Boolean logic combination of gated cell records. At block 760b, the method 700b includes displaying an indication of the sets of normalized cell counts, ordered according to the clustering. At block 770b, the method 700b includes receiving a user indication of at least one biomarker across the cells samples. At block 780b, the method 700b includes, using the sets of normalized cell counts, training a classifier to predict the at least one biomarker for a cell sample based on an input set of normalized cell counts determined from the cell sample.

The method 700b can include additional or alternative steps or features.

Referring now to FIG. 7C, a method 700c is illustrated, optionally using the computing device of FIGS. 5-6. Method 700c includes, at block 710c, obtaining a plurality of cell records for a target cell sample, wherein each cell record of the plurality of cell records represents a set of optical properties detected from a respective cell of the target cell sample. Then, at block 720c, the method 700c includes obtaining a plurality of sets of one or more threshold values, wherein a given set of one or more threshold values separates the plurality of cell records into at least two populations with respect to a respective optical property of the set of optical properties. Next, at block 730c, the method 700c additionally includes determining a set of cell counts for the target cell sample by determining, for each possible cell class separated by the threshold values, a respective count of cells of the target cell sample that fall within the respective cell class. The method 700c then includes normalizing the set of cell counts to generate a set of normalized cell counts, at block 740c. At block 750c, the method 700c includes applying the normalized cell counts to a trained classifier to predict a biomarker for the target cell sample.

The method 700c can include additional or alternative steps or features.

As discussed above, a non-transitory computer-readable medium having stored thereon program instructions that upon execution by a processor (e.g., 202) may be utilized to cause performance of any of functions of the foregoing methods.

### IV. Conclusion

The above detailed description describes various features and functions of the disclosed systems, devices, and methods with reference to the accompanying figures. In the figures, similar symbols typically identify similar components, unless the context indicates otherwise. The illustrative embodiments described in the detailed description, figures, and claims are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A system comprising:
at least one processor; and
a non-transitory computer-readable medium, having stored therein instructions executable by the at least one processor to cause the system to perform operations comprising:
obtaining a plurality of cell records for a plurality of cell samples, wherein each cell record of the plurality of cell records represents a set of optical properties detected from a respective cell of one of the cell samples such that each cell sample is associated with a respective non-overlapping subset of the cell records;
for each optical property in the set of optical properties, determining, based on the set of cell records, a respective at least one threshold value that separates the plurality of cell records into at least two populations with respect to the respective optical property;
determining, for each cell sample, a respective set of cell counts, wherein determining a set of cell counts for a particular cell sample comprises determining, for each possible cell class separated by the threshold values, a respective count of cells of the particular cell sample that fall within the respective cell class; and
for each cell sample, normalizing the respective set of cell counts to generate a respective set of normalized cell counts.

2. The system of claim 1, wherein the operations further comprise:
based on the sets of normalized cell counts, clustering at least one of (i) the cell samples according to similarity with respect to their respective sets of normalized cell counts, or (ii) the cell classes according to similarity with respect to pattern of normalized cell counts across cell samples for each cell class; and
displaying an indication of the sets of normalized cell counts, ordered according to the clustering.

3. The system of claim 2, wherein the operations further comprise, prior to displaying the indication, further normalizing the sets of normalized cell counts within each of the cell classes.

4. The system of claim 2, wherein the operations further comprise:
receiving a user indication of one or more subsets of samples within the plurality of cell samples; and
using the sets of normalized cell counts, training a classifier to predict membership of a cell sample within the one or more subsets of samples based on an input set of normalized cell counts determined from the cell sample.

5. The system of claim 2, wherein the operations further comprise, prior to displaying the indication, removing, from the cell classes, any of the cell classes that are represented in any of the cell samples by a cell count that exceed a threshold value.

6. The system of claim 2, wherein clustering at least one of (i) the cell samples according to similarity with respect to their respective sets of normalized cell counts, or (ii) the cell classes according to similarity with respect to pattern of normalized cell counts across cell samples for each cell class comprises performing a hierarchical clustering.

7. The system of claim 1, wherein the operations further comprise:
obtaining a set of labels that identify one or more subsets of samples within the plurality of cell samples; and
using the sets of normalized cell counts and sets of labels, training a classifier to predict membership of a target cell sample within the one or more subsets of samples based on an input set of normalized cell counts determined from the target cell sample.

8. The system of claim 7, wherein the operations further comprise, prior to training the classifier, further normalizing the sets of normalized cell counts within each of the cell classes.

9. The system of claim 7, wherein the operations further comprise, prior to training the classifier, removing, from the cell classes, any of the cell classes that are represented in any of the cell samples by a cell count that is less than a threshold value.

10. The system of claim 7, wherein the operations further comprise, prior to training the classifier and based on the sets of normalized cell counts, clustering at least one of (i) the cell samples according to similarity with respect to their respective sets of normalized cell counts, or (ii) the cell classes according to similarity with respect to pattern of normalized cell counts across cell samples for each cell class.

11. The system of claim 1, wherein determining, based on the set of cell records, a respective at least one threshold value that separates the plurality of cell records into at least two populations with respect to the respective optical property comprises, for a particular optical property of the set of optical properties, determining at least two threshold values that separates the plurality of cell records into at least three populations with respect to the particular optical property.

12. The system of claim 1, wherein a first optical property of the set of optical properties represents an intensity of light at a first wavelength that is received from a cell in response to the cell being illuminated by light at a second wavelength, and wherein a second optical property of the set of optical properties represents an intensity of light at the first wavelength that is received from a cell in response to the cell being illuminated by light at a third wavelength that differs from the second wavelength.

13. The system of claim 1, further comprising a high-throughput flow cytometer, wherein the operations further comprise operating the high-throughput flow cytometer to obtain the plurality of cell records for the plurality of cell samples.

14. A system comprising:
at least one processor; and
a non-transitory computer-readable medium, having stored therein instructions executable by the at least one processor to cause the system to perform operations comprising:
obtaining a plurality of cell records for a plurality of cell samples, wherein each cell record of the plurality of cell records represents a set of optical properties detected from a respective cell of one of the cell samples such that each cell sample is associated with a respective non-overlapping subset of the cell records;
for two or more digital gates, digitally gating, based on the set of cell records, the plurality of cell records into at least two populations with respect to at least one of the optical properties;
determining, for each cell sample, a respective set of cell counts, wherein determining a set of cell counts for a particular cell sample comprises determining, for each possible Boolean logical combination of gated cell records across each of the two or more digital gates, a respective count of cells of the particular cell sample that fall within the respective Boolean logical combination of gated cell records;
for each cell sample, normalizing the respective set of cell counts to generate a respective set of normalized cell counts;
based on the sets of normalized cell counts, clustering at least one of (i) the cell samples according to similarity with respect to their respective sets of normalized cell counts, or (ii) the Boolean logic combinations of gated cell records according to similarity with respect to pattern of normalized cell counts across cell samples for each Boolean logic combination of gated cell records;
displaying an indication of the sets of normalized cell counts, ordered according to the clustering;
receiving a user indication of at least one biomarker across the cells samples; and
using the sets of normalized cell counts, training a classifier to predict the at least one biomarker for a cell sample based on an input set of normalized cell counts determined from the cell sample.

15. (canceled)

16. A system comprising:
at least one processor; and
a non-transitory computer-readable medium, having stored therein instructions executable by the at least one processor to cause the system to perform operations comprising:
obtaining a plurality of cell records for a target cell sample, wherein each cell record of the plurality of cell records represents a set of optical properties detected from a respective cell of the target cell sample;
obtaining a plurality of sets of one or more threshold values, wherein a given set of one or more threshold values separates the plurality of cell records into at least two populations with respect to a respective optical property of the set of optical properties;
determining a set of cell counts for the target cell sample by determining, for each possible cell class separated by the threshold values, a respective count of cells of the target cell sample that fall within the respective cell class;
normalizing the set of cell counts to generate a set of normalized cell counts; and
applying the normalized cell counts to a trained classifier to predict a biomarker for the target cell sample.

17. The system of claim 16, wherein the operations further comprise, prior to applying the normalized cell counts to the trained classifier, removing, from the cell classes, a pre-specified subset of the cell classes.

18. The system of claim 16, wherein at least one set of one or more threshold values of the plurality of sets of one or more threshold values comprises at least two threshold values that separate the plurality of cell records into at least three populations with respect to a respective at least one optical property of the set of optical properties.

19. The system of claim 16, wherein a first optical property of the set of optical properties represents an intensity of light at a first wavelength that is received from a cell in response to the cell being illuminated by light at a second wavelength, and wherein a second optical property of the set of optical properties represents an intensity of light at the first wavelength that is received from a cell in response to the cell being illuminated by light at a third wavelength that differs from the second wavelength.

20. The system of claim 16, further comprising a high-throughput flow cytometer, wherein the operations further comprise operating the high-throughput flow cytometer to obtain the plurality of cell records for the plurality of cell samples.

21. The system of claim 16, wherein the trained classifier has been trained using the method of claim 1.

22-23. (canceled)
